(19) United States
(12) Patent Application Publication
(10) Pub. No.: US 2020/0093611 A1
(43) Pub. Date: Mar. 26, 2020

(54) ROBOTIC IMPLANT INSERTION SYSTEM WITH FORCE FEEDBACK TO IMPROVE THE QUALITY OF IMPLANT PLACEMENT AND METHOD OF USE THEREOF

(71) Applicant: THINK SURGICAL, INC., Fremont, CA (US)

(72) Inventors: Micah Forstein, Fremont, CA (US); Steve Whiseant, Fremont, CA (US)

(21) Appl. No.: 16/578,708

(22) Filed: Sep. 23, 2019

Related U.S. Application Data
(60) Provisional application No. 62/736,051, filed on Sep. 25, 2018.

(51) Int. Cl. A61F 2/46; A61B 17/16

(52) U.S. Cl. CPC A61F 2/46; A61B 17/16; A61F 2002/4632; A61B 2017/1602; A61F 2002/4633; A61F 2002/4622; A61F 2002/4666

(57) ABSTRACT

A method and system are provided that assesses the quality of implant placement intraoperatively, determines the accuracy of a prepared implant cavity, and alerts a user of an impending bone fracture during computer-assisted orthopedic surgery. The method and system improve the quality of implant placement in a bone compared to conventional techniques by utilizing force feedback acquired while robotically inserting the implant in the bone.

### RELATED APPLICATIONS

This application claims priority benefit of U.S. Provisional Application Ser. No. 62/736,051 filed 25 Sep. 2018, the contents of which are hereby incorporated by reference.

### TECHNICAL FIELD

The present invention generally relates to the field of computer-assisted orthopedic surgery, and in particular, to a system and method to improve the quality of implant placement in a bone compared to conventional techniques by utilizing force feedback acquired while robotically inserting the implant in the bone.

### BACKGROUND

Throughout a lifetime, bones and joints become damaged and worn through normal use, disease, and traumatic events. Arthritis is a leading cause of joint damage, which can cause cartilage degradation, pain, swelling, stiffness, and bone loss over time. If the pain associated with the dysfunctional joint is not alleviated by less-invasive therapies, the joint may need to be replaced with a procedure called total joint arthroplasty (TJR). TJR is an orthopedic surgical procedure in which the typically worn articular surfaces of the joint are replaced with prosthetic components, or implants. TJR typically requires the removal of the articular cartilage of the joint including a varying amount of bone. This cartilage and bone is then replaced with synthetic implants, typically metal and plastic, which form the new synthetic joint surfaces.

The accurate placement and alignment of the implants on the bone is a large factor in determining the success of a TJR procedure. A slight misalignment may result in poor wear characteristics, reduced functionality, poor clinical outcomes, and decreased longevity. Therefore, several TJR procedures are now performed with computer-assistance, and even more advanced procedures utilize robotic surgical systems. One such robotic surgical system is the TSOLUTION ONE® Surgical System (THINK Surgical, Inc., Fremont, Calif.), which aids in the planning and execution of total hip arthroplasty (THA) and total knee arthroplasty (TKA). The TSOLUTION ONE® Surgical System includes: a pre-operative planning software program to generate a surgical plan using an image data set and/or three dimensional (3-D) models of the patient's bone and computer-aided design (CAD) models of several implants; and an autonomous surgical robot that precisely mills the bone to receive an implant according to the surgical plan.

With regard to pre-operative planning, prior art FIGS. 1A-1C depict a bone model (BM), as shown in prior art FIG. 1A, having an implant model IM positioned therein. The user may adjust the model, size, position and orientation (POSE) of the implant model IM, as shown in FIG. 1B, relative to the bone model BM to designate the best fit, fill, and/or POSE for an implant with a bone B. The model, size, and POSE of the implant model IM in the bone model BM also designates the POSE and dimensions for an implant cavity IC, as shown in FIG. 1C, to be made in the bone B to receive the implant. For surgical robotic procedures, a set of instructions, such as a cut-file CF (e.g., a set of cut-paths and end-effector velocities/accelerations) or a set of boundaries may be pre-defined relative to the implant model IM to instruct the robotic system to create the implant cavity IC to precisely receive the implant in the planned POSE. The set of instructions may further include regions to be cut (e.g., the dotted region above the implant referred to as the pre-cut PC) to gain access to create the implant shape in the planned POSE. The pre-operative planning data is saved and transferred to the surgical robot in the operating room to create the implant cavity IC according to the plan. Once the bone is prepared, the surgeon installs the implant in the implant cavity IC.

One problem existing with this conventional approach is determining how well the implant fits in the bone B once installed. Conventional methods rely on post-operative x-rays to determine the quality of the implant placement (e.g., fit, fill, and/or alignment of the implant in the bone B). Post-operative x-ray imaging precludes a surgeon from improving the quality of the implant placement. Intra-operative x-rays are possible, but at the expense of additional radiation exposure, increased procedure time in the operating room, and associated additional costs.

Another problem is determining the accuracy of the prepared implant cavity IC before or during implant insertion. Slight errors in the system may accumulate (e.g., registration error, calibration error) and affect the preparation of the implant cavity IC slightly. These errors may affect the quality of the implant placement.

Further, in patient cases with compromised bone, bone fractures are possible while inserting and setting the implant in the bone. Currently, there is no feedback to alert the surgeon of an impending fracture during implant insertion.

Thus, there exists a need for a system and method to assess the quality of implant placement intraoperatively, determine the accuracy of a prepared implant cavity, and alert a user of an impending bone fracture during orthopedic surgery.

### SUMMARY

A method is provided that assesses the quality of implant placement intra-operatively with a robotic surgical system. The method includes generating a surgical plan having: a position and orientation (POSE) of a cavity to be created in a bone to receive an implant, and trajectory parameters for robot insertion of the implant. The method includes the steps of creating a cavity in the bone, robotically inserting the implant into the created cavity while recording force data, compiling the force data, in real-time, to generate an actual force profile representing the forces experienced on the implant during insertion, and comparing the actual force profile to an expected force profile model to determine at least one of: an accuracy of the created cavity, a quality of fit of the implant in the cavity, or an impending bone fracture.

A robotic surgical system is provided to assess the quality of implant placement intra-operatively. The system includes a surgical robot having a manipulator arm, an end-effector attached to a distal end of the manipulator arm, an implant attached to the end-effector; a surgical plan having trajectory parameters to guide the manipulator arm to robotically insert the implant into a cavity, and a force sensor to sense forces generated on the implant during robotic insertion. The system further includes a computer having a processor and memory. The memory having an expected force profile model stored therein in communication with a comparison module that when executed by the processor causes the processor to build an actual force profile from force data collected from the force sensor during robotic insertion and compares the actual force profile to the expected force profile model to determine at least one of an accuracy of the created cavity; a quality of fit of the implant in the bone; or an impending bone fracture.

### BRIEF DESCRIPTION OF THE DRAWINGS

The present invention is further detailed with respect to the following drawings that are intended to show certain aspects of the present of invention, but should not be construed as a limit on the practice of the invention, wherein:

FIGS. 1A-1C illustrate prior art views of a bone model with an implant model therein (FIG. 1A), adjustment of the position and orientation of the implant model (FIG. 1B), and the position and orientation of the implant cavity (FIG. 1C);

FIG. 2 is a flow chart of a method to assess the quality of implant placement intraoperatively, determine the accuracy of a prepared implant cavity, and alert a user of an impending bone fracture during orthopedic surgery in accordance with embodiments of the invention;

FIGS. 3A to 3C depict an embodiment of an end-effector of a robotic system inserting an implant, into an implant cavity IC prepared in the bone B, where FIG. 3A depicts a perfectly prepared implant cavity IC, FIG. 3B depicts an implant cavity IC having a slight deviation D from the planned implant cavity, and FIG. 3C depicts a bone having an impending bone fracture IF;

FIG. 4 is a flow chart of a method to build an expected force profile model in accordance with embodiments of the invention;

FIG. 5 illustrates a three-dimensional force profile in accordance with embodiments of the invention;

FIG. 6 depicts a plurality of force profiles from several mock bone cases to build an expected force profile model in accordance with embodiments of the invention; and

FIG. 7 shows an example of a computer-assisted surgical device in the context of an operating room (OR) for implanting embodiments of the invention.

### DETAILED DESCRIPTION

The present invention has utility as a system and method to assess the quality of implant placement intraoperatively, determine the accuracy of a prepared implant cavity, and alert a user of an impending bone fracture during orthopedic surgery. The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular embodiment may be deleted from that embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations and variations thereof.

Further, it should be appreciated that the systems and methods described herein may be applied to bones and joints in the body illustratively including the knee, ankle, elbow, wrist, skull, and spine, as well as revision of initial repair or replacement of any of the aforementioned bones or joints.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents, and other references mentioned herein are incorporated by reference in their entirety.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also, as used herein, “and/or” refers to and encompasses any and all possible combinations of one or more of the associated listed items, as well as the lack of combinations when interpreted in the alternative (“or”).

As used herein, the term “digitizer” refers to a device capable of measuring, defining, and/or designating physical coordinates in three-dimensional space. For example, the ‘digitizer’ may be: a “mechanical digitizer” having passive links and joints, such as the high-resolution electro-mechanical sensor arm described in U.S. Pat. No. 6,033,415; a non-mechanically tracked digitizer probe (e.g., optically tracked, electromagnetically tracked, acoustically tracked, and equivalents thereof) as described for example in U.S. Pat. No. 7,043,961; or an end-effector of a robotic device.

As used herein, the term “digitizing” refers to the collecting, measuring, and/or recording of physical points in space with a digitizer.

As used herein, the term “pre-operative bone data” refers to bone data used to pre-operatively plan a procedure before making modifications to the actual bone. The pre-operative bone data may include one or more of the following. A patients actual exposed bone prior to modification, an image data set of a bone, a virtual generic bone model, a physical bone model, a virtual patient-specific bone model, or a set of data collected directly on a bone intra-operatively commonly used with imageless computer-assist devices.

As used herein, the term “registration” refers to the determination of the POSE and/or coordinate transformation between two or more objects or coordinate systems such as a computer-assist device, a bone, pre-operative bone data, surgical planning data (i.e., an implant model, cut-file, virtual boundaries, virtual planes, cutting parameters associated with or defined relative to the pre-operative bone data), and any external landmarks (e.g., a fiducial marker array) associated with the bone, if such landmarks exist. Methods of registration known in the art are described in U.S. Pat. Nos. 6,033,415, 8,010,177, and 8,287,522.

As used herein, the term “real-time” refers to the processing of input data within milliseconds such that calculated values are available within 10 seconds of computational initiation.

Also described herein are ‘computer-assisted surgical devices’. A computer assisted surgical device refers to any device/system requiring a computer to aid in a surgical procedure. Examples of a computer-assisted surgical device include a tracking system, tracked passive instruments, active or semi-active hand-held surgical devices and systems, autonomous serial-chain manipulator systems, haptic serial chain manipulator systems, parallel robotic systems, or master-slave robotic systems, as described in U.S. Pat. Nos. 5,086,401; 7,206,626; 8,876,830; 8,961,536; 9,707,043; and PCT. Pub. WO/2016/049180.

While the present invention is illustrated visually hereafter with respect to a proximal femur as an example of the target bone for which the present invention is applied, it is appreciated that the present invention is equally applicable to other bones of a human, non-human primate, or other mammals.

Furthermore, it should be appreciated that while the systems and methods described herein are with reference to total hip arthroplasty (THA) implants, any of a wide variety of different bone implants may likewise utilize the teachings of the present invention (e.g., a knee implant, shoulder implant, ankle implant, wrist implant, skull, and spinal implant) as well as revision of initial repair or replacement of any of the aforementioned bones or joints.

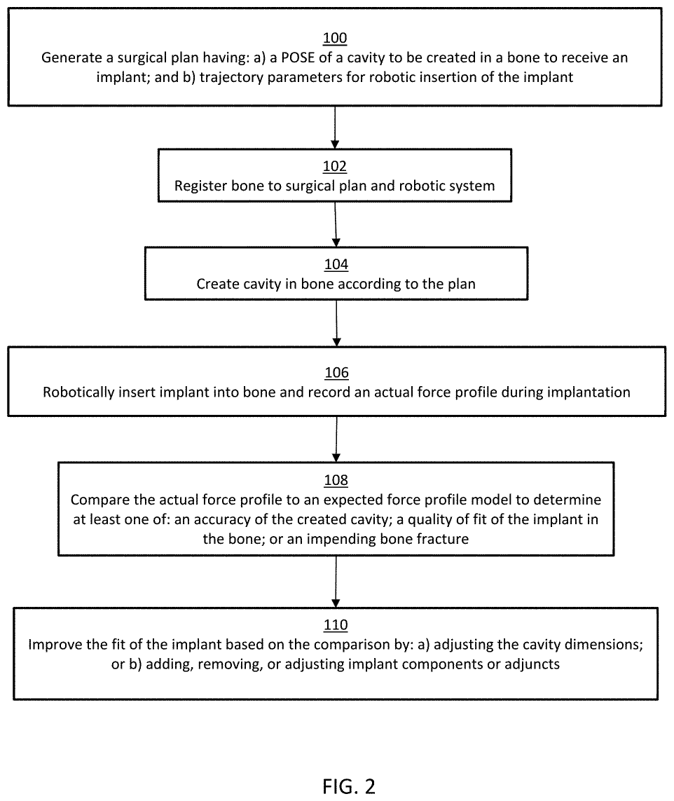

With reference now to the drawings, FIG. 2 depicts an embodiment of a method to assess the quality of implant placement intraoperatively, determine the accuracy of a prepared implant cavity, and alert a user of an impending bone fracture during orthopedic surgery. The method includes the following. A surgical plan is generated having: a) a POSE of a cavity to be created in a bone to receive an implant; and b) trajectory parameters for robotic insertion of the implant (Block 100). The bone is registered to the surgical plan and the robotic surgical system (Block 102). An implant cavity is created according the surgical plan (Block 104). The robotic surgical system inserts the implant into the bone and records an actual force profile during insertion (Block 106). The actual force profile is compared to an expected force profile model to determine at least one of: an accuracy of the created cavity; a quality of the implant placement in the bone; or an impending bone fracture (Block 108). The fit of the implant in the bone may be improved based on the comparison of the actual profile and expected profile by: a) adjusting the cavity dimensions; b) adding, removing, or adjusting implant components or adjuncts (Block 110). An impending bone fracture may further be mitigated based on the comparison of the actual force profile and expected force profile. Details of the above embodiments are further described below.

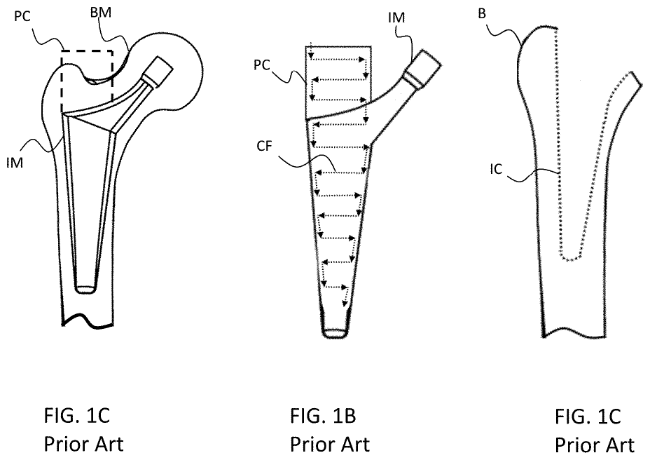

The user may generate a surgical plan by planning the POSE of an implant model IM relative to pre-operative bone data in a pre-operative planning software program having a graphical user interface (GUI) (Block 100). In a particular inventive embodiment, the pre-operative bone data is a virtual three-dimensional (3-D) bone model generated from an image data set of a subject's anatomy. The image data set may be collected with an imaging modality such as computed tomography (CT), dual-energy x-ray absorptiometry (DEXA), magnetic resonance imaging (MRI), X-ray scans, ultrasound, or a combination thereof. The 3-D bone model(s) are readily generated from the image data set using medical imaging software such as Mimics® (Materialise, Plymouth, Mich.) or other techniques known in the art such as the one described in U.S. Pat. No. 5,951,475. A set of 3-D computer aided design (CAD) models of the manufacturer's implants (implant models) are pre-loaded in the software that allows the user to place the components of a desired implant to the 3-D bone model of the boney anatomy to designate the best fit, position and orientation of the implant to the bone. The planning software program may further allow the user to custom design implants on/around the 3-D model(s). In other embodiments, the user may plan the position for an implant directly on the image date set and may use bone quality data to aid in planning the implant position. The planned POSE for the implant model relative to the data set and/or 3-D bone model engenders the POSE of the cavity to be created in the bone to receive the implant in the planned POSE as described above with reference to FIGS. 1A-1C. In other embodiments, the POSE of the cavity may be planned based on the bone itself using the contours between the trabecular and cortical bone as seen in standard CT image slices.

The surgical plan further includes trajectory parameters for robotic insertion of the implant. Based on at least one of the POSE of the cavity, the one or more dimensions of the cavity, and one or more dimensions of the implant, a trajectory to insert the implant in the bone may be determined. In one embodiment of the present invention, one or more trajectories may be defined that align one or more regions of the implant (e.g., distal tip, lateral/medial edge/surface, anterior/posterior edge/surface) to its corresponding final resting place in the cavity as defined by the user's placement of the implant model IM relative to the bone model BM. In other embodiments, the trajectory may be defined by aligning a specific reference point on the implant with a specific landmark on the anatomy (e.g., greater trochanter, epicondyles) as defined by the planned POSE of the implant model IM relative to the bone model BM. These trajectory parameters may be in the form of a set of position and/or orientation vectors, or points, that are defined relative to the pre-operative bone data (e.g., bone model BM) in the surgical plan. The parameters may further include a set of velocities and/or accelerations in which to insert the implant in the bone. Once the bone is registered, the robot can align the implant and insert the implant in the bone according to the trajectory parameters.

The final surgical plan having the POSE of the cavity and the trajectory parameters may be saved and/or transferred to the computer-assist device in the operating room to assist the surgeon with the orthopedic procedure.

For a computer-assisted surgical procedure, the bone is exposed in the operating room using techniques known in the art. Once exposed, at least one of the bone, the pre-operative bone data, surgical planning data, and any landmarks associated with the bone are registered to the computer-assist device (Block 102). For an imageless computer-assist device, the user may collect several points on a tracked bone to create a point cloud representation of the bone to register to the computer-assist device without pre-operative images. For image-guided computer-assist devices, registration of the bone with pre-operative images may be performed with aforementioned techniques known in the art (e.g., point-to-point, point-to-surface, image registration). This registration also registers the trajectory parameters to the bone and the robotic system.

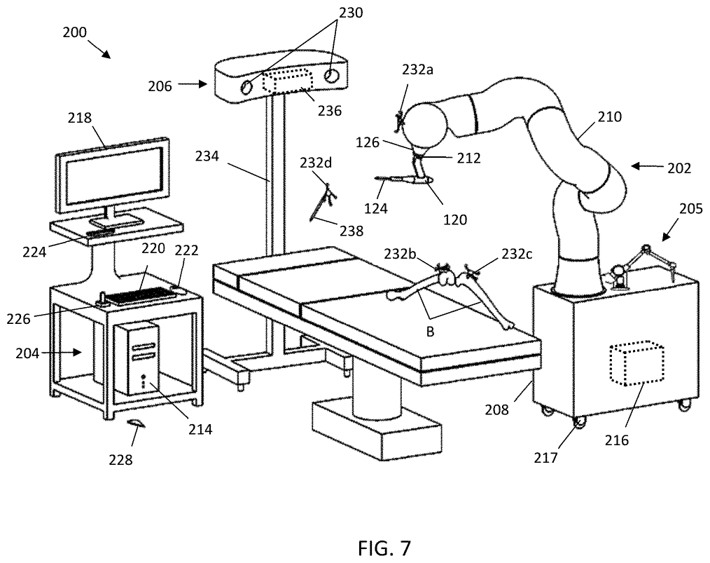

Subsequently, the bone is prepared (i.e., cut, shaved, or otherwise modified) to receive an implant according to the surgical plan (Block 104). The bone may be prepared using a variety of different tools, either manual tools (e.g., broaches, cut-guides, reamers) or a computer-assist device. Examples of computer-assisted devices include: a tracking system (e.g., an optical tracking system, mechanical tracking system) for tracking one or more tools or a simple probe; a 1-6+ degree of freedom or more hand-held surgical system; an autonomous serial-chain manipulator system; a haptic serial-chain manipulator system; a parallel robotic system; a robot mounted to the bone of the subject; a robotically maneuvered cut-guide; a tracked or navigated saw, broach, reamer, implant, or cut-guide; a master-slave robotic system; or any combination thereof. Such systems are described in U.S. Pat. Nos. 5,086,401; 6,757,582; 7,206,626; 8,560,047; 8,571,638; 8,876,830; 8,961,536; 9,283,048; and 9,707,043; and PCT Pub. No. WO/2016/049180. In a specific embodiment, a robotic system as described with reference to FIG. 7 automatically mills the bone according to a cut-file CF. In other embodiments, the bone is prepared with a haptic robotic surgical system that haptically restrains a user wielding an end-effector inside a set of defined boundaries.

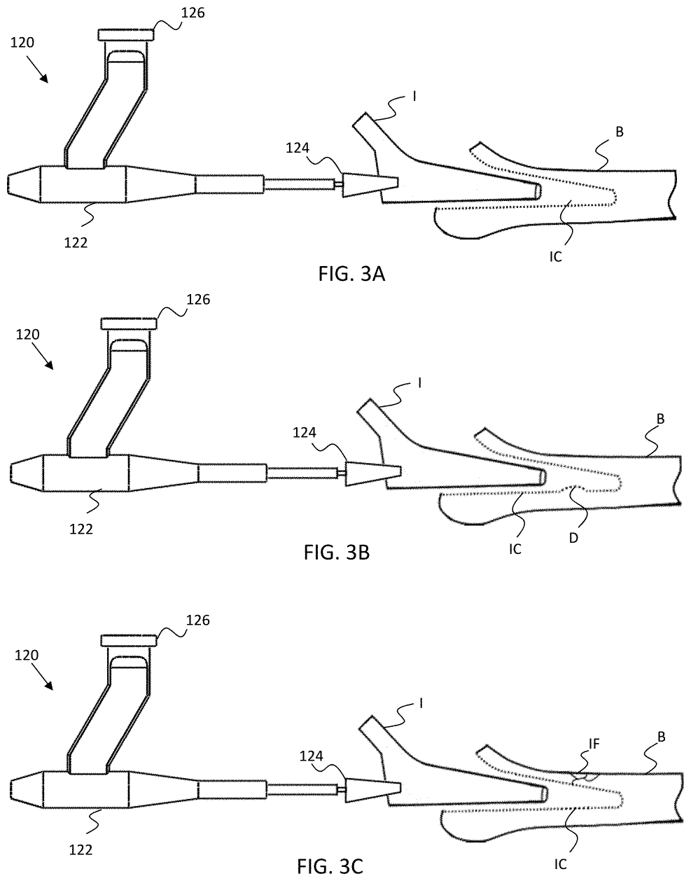

After the bone is prepared, a robotic system robotically inserts an implant in/on the prepared bone while acquiring force data (Block 106). FIGS. 3A to 3C depict an example of an end-effector 120 of a robotic system inserting an implant, I into an implant cavity IC prepared in the bone B, where FIG. 3A depicts a perfectly prepared implant cavity, IC. FIG. 3B depicts an implant cavity IC having a slight deviation D from the planned implant cavity, and FIG. 3C depicts a bone having an impending bone fracture, IF. The robotic system includes an end-effector 120 attached to a distal end of a robotic arm, and a force sensor 126 as further described below with reference to FIG. 7. The end-effector 120 is generally configured to hold, grasp, attach, or connect with an implant I. In a particular inventive embodiment, the end-effector 120 includes a tool holder 122 that connects with an implant holder 124. The implant holder 124 is configured to hold, grasp, or otherwise attach or connect with an implant, I. For example, the implant holder 124 may be: a pair of jaws, clamps, or fingers for grasping the implant, I; a magnet to magnetically couple with the implant I; a vacuum; an adhesive to stick to the implant I; a screw or pin that assembles with a corresponding feature on the implant I; or other fastening element or coupler. The tool holder 122 may improve the versatility of the end-effector 120 by allowing different tools to assemble with the tool holder 122 without having to change or swap-out the end-effector 120 during a procedure. For example, during the preparation of the implant cavity IC, the tool holder 122 may house a motor that rotates an end-mill to mill out the implant cavity IC. Then, during robot insertion of the implant I, the end-mill may be removed from the tool holder 122 and an implant holder 124 may be assembled thereto to attach/connect with an implant, I for insertion with the implant cavity IC. In other embodiments, there is no tool holder 122 wherein the end-effector 120 itself is an implant holder 124 assembled directly to the distal end of a robotic arm.

The force sensor 126 is configured to sense the forces imposed on the end-effector 120 and more particularly to sense the forces imposed on the implant, I during robotic insertion. The force sensor 126 may be disposed on a distal end of the robotic arm, the end-effector 120, or on a link or joint of the robotic arm. In particular embodiments, the force sensor 126 is a 6-degree-of-freedom (6-DOF) force sensor.

To robotically insert the implant, I into the implant cavity IC, the implant, I is first assembled to the end-effector 120. The POSE of the implant, I is then determined relative to the robotic system such that the robot knows the POSE of the implant, I relative to the trajectory parameters already registered to the robotic system. The implant, I may be registered to the robotic system by digitizing several landmarks on the implant, I such as the distal tip, the neck, the tibial plateau, or other defining characteristics of the implant, I. In other embodiments, a model of the implant is stored in the robotic system, wherein several points on the implant, I are collected and matched with corresponding points or surfaces on the model of the implant. In a further embodiment, the implant, I may have an attachment site that assembles with the implant holder 124 in a known POSE. In another embodiment, a fiducial marker array is assembled to the implant, I to permit a tracking system to track the POSE of the implant, I relative to the robotic system. In a further embodiment, the implant is 3-D scanned with a tracked 3-D scanner (e.g., laser scanner). Once the POSE of the implant, I is known relative to the robotic system and therefore the trajectory parameters, the implant, I is robotically inserted into the implant cavity IC. During the insertion, the force sensor 126 acquires force data. The robotic system compiles the force data into an actual force profile which represents all of the forces imposed on the implant, I during the insertion process.

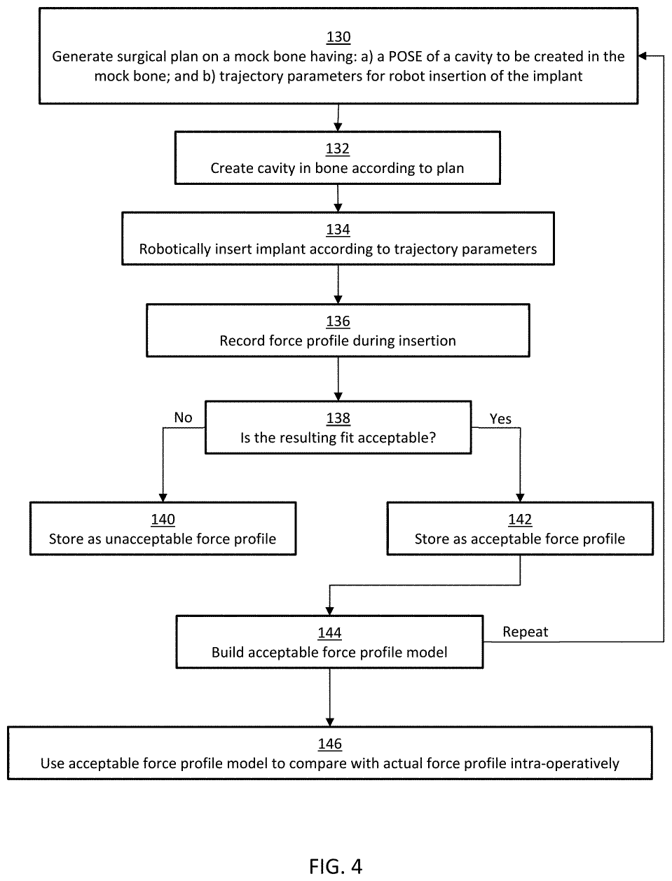

The actual force profile is compared to an expected force profile model to determine at least one of: an accuracy of the created cavity; a quality of fit of the implant in the bone; or an impending bone fracture (Block 108). With reference to FIG. 4, an embodiment of a method to build an expected force profile model is illustrated. The method includes the following. A surgical plan is created on a mock bone such as a cadaver bone or other material having characteristics of real bone. The final surgical plan includes: a) a POSE of a cavity to be created in the mock bone; and b) trajectory parameters for robot insertion of the implant (Block 130). The mock bone is prepared using similar techniques as described above to create the cavity in the mock bone (Block 132). An implant is robotically inserted into the cavity according to the trajectory parameters (Block 134). The same methods as described above with reference to (Block 106) may be used to robotically insert the implant into the mock bone. During robotic insertion, force data is recorded (Block 136), wherein the robotic system compiles the force data into a force profile which represents all of the forces imposed on the implant during the insertion process. The force profiles are saved and experiments/tests are subsequently conducted on the implant in the mock bone. The testing may include longevity testing, micro-motion studies, imaging scans, load testing, stress analysis, fatigue testing, finite element analysis with the data from the imaging scans, as well as other performance tests if required. These tests may be conventional studies conducted by implant manufacturer's to determine the longevity and integrity of their implants prior to their release in the clinical field. Pass/fail criteria is established for the tests to determine if the resulting fit of the implant in the mock bone is acceptable (Block 138). For example, micro-motion studies may be performed. If there is micro-motion of the implant in the mock bone greater than threshold amount, then the resulting fit is unacceptable. In this case, the force profile resulting from this particularly created cavity (Block 132) and robot insertion (Block 134) is labeled unacceptable (Block 140) and may either be discarded or used to build an expected force profile model indicative of a poorly fitted implant. On the other hand, if the micro-motion studies are positive, the force profile is stored as an acceptable force profile (Block 142) and used to build an expected force profile model indicative of a well fitted implant (i.e., good quality). The method is repeated for several mock bone cases to build the expected force profile model. Several different force profile models may be generated for a particular implant model, size, and/or shape, or for a given cavity to be created for comparison. Force profile models may further be generated indicative of impending bone fractures. The expected force profile model is then compared to the actual force profile as described above (Block 146). Thus, based on a similarity between the actual force profile and an expected force profile model at least one of the following may be determined: an accuracy of the created cavity; a quality of fit of the implant in the bone; or an impending bone fracture (Block 108).

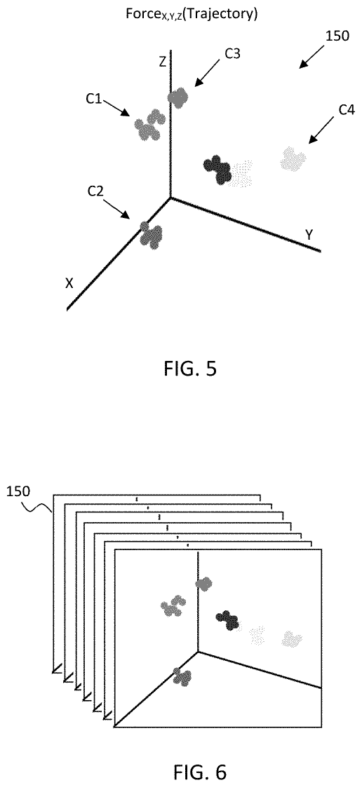

FIG. 5 depicts an example of a force profile 150 for a single case, either on a mock bone or a real bone. The forces on the implant during insertion are collected as a function of the implant path/trajectory in the bone. The clusters (C1, C2, C3, C4) illustratively represent the forces collected for a given trajectory position (e.g., a first set of forces C1 acquired when the implant is at a first position in the cavity, a second set of forces C2 acquired when the implant is at a second position in the cavity, etc.). All of the forces collected during insertion are stored to build this force profile 150. FIG. 6 depicts a plurality of force profiles 150 from several mock bone cases to build the expected force profile model. Techniques know in the art may be used to build the model such as least-squares or regression.

Based on the comparison of the actual force profile and the expected force profile model, the user may have several options to improve the quality of the implant placement, if required (Block 110). If the comparison indicates the created cavity has a slight deviation D (as shown in FIG. 3B) from the plan, the user may attempt to adjust the cavity dimensions to fix the deviation D. The comparison may automatically determine where the deviation D is located such that the surgical system can indicate as such to the user. The user may manually adjust the cavity dimensions, or use computer/robotic assistance. In another embodiment, the user may have the option to add, remove, or adjust the implant or adjuncts thereto. The implant may have modular components where the user can adjust a position or size of one of the modular components to improve the quality of the implant placement. The user may be able to add shims, cement, or other tertiary components to the bone and/or implant to improve the quality of the placement. The surgical system may provide instructions or recommendations to the user on how to improve the quality of the implant placement. Once the user has made the necessary improvements, the implant is seated and the surgical procedure is complete. As a result, a surgeon is able to take steps previously unavailable during the course of the procedure to avoid bone fracture and improve implant fit.

In a specific inventive embodiment, an impending bone fracture may be determined by monitoring the force data during robotic insertion and if the forces exceed a threshold force, the insertion may be paused and the user warned about a potential bone fracture. Thus, a comparison of a force profile isn't necessarily needed to warn the user of an impending bone fracture.

Surgical System

With reference to FIG. 7, an inventive embodiment of a robotic surgical system 200 is shown capable of implementing embodiments of the inventive method described above. The surgical system 200 generally includes a surgical robot 202, a computing system 204, and may include at least one of a mechanical digitizer arm 205 or a tracking system 206.

The surgical robot 202 may include a movable base 208, a manipulator arm 210 connected to the base 208, an end-effector 120 located at a distal end 212 of the manipulator arm 210, and a force sensor 126 positioned proximal to the end-effector 120 for sensing forces experienced on the end-effector 120. The base 208 includes a set of wheels 217 to maneuver the base 208, which may be fixed into position using a braking mechanism such as a hydraulic brake. The base 208 may further include an actuator to adjust the height of the manipulator arm 210. The manipulator arm 210 includes various joints and links to manipulate the end-effector 120 in various degrees of freedom. The joints are illustratively prismatic, revolute, spherical, or a combination thereof.

The computing system 204 generally includes a planning computer 214; a device computer 216; a tracking computer 236 if a tracking system 206 is present; and peripheral devices. The planning computer 214, device computer 216, and tracking computer 236, may be separate entities, single units, or combinations thereof depending on the surgical system. The peripheral devices allow a user to interface with the surgical system components and may include: one or more user-interfaces, such as a display or monitor 218 for the graphical user interface (GUI); and user-input mechanisms, such as a keyboard 220, mouse 222, pendent 224, joystick 226, foot pedal 228, or the monitor 218 in some inventive embodiments has touchscreen capabilities.

The planning computer 214 contains hardware (e.g., processors, controllers, and/or memory), software, data and utilities that are in some inventive embodiments dedicated to the planning of a surgical procedure, either pre-operatively or intra-operatively. This may include reading medical imaging data, segmenting imaging data, constructing three-dimensional (3D) virtual models, storing computer-aided design (CAD) files, providing various functions or widgets to aid a user in planning the surgical procedure, and generating surgical plan data. The planning computer is further programmed to determine robot insertion trajectory parameters based on the desired POSE for an implant in a bone. The final surgical plan may include image data, patient data, registration data, implant position data, trajectory parameters, and/or operational data. The operational data may be a set of instructions for modifying a volume of tissue that is defined relative to the anatomy, such as a set of cutting parameters (e.g., cut paths, velocities) in a cut-file to autonomously modify the volume of bone, a set of virtual boundaries defined to haptically constrain a tool within the defined boundaries to modify the bone, a set of planes or drill holes to drill pins in the bone, a graphically navigated set of instructions for modifying the tissue, and the trajectory parameters for robotic insertion of an implant. In particular inventive embodiments, the operational data specifically includes a cut-file for execution by a surgical robot to autonomously modify the volume of bone, which is advantageous from an accuracy and usability perspective. The surgical plan data generated from the planning computer 214 may be transferred to the device computer 216 and/or tracking computer 236 through a wired or wireless connection in the operating room (OR); or transferred via a non-transient data storage medium (e.g., a compact disc (CD), a portable universal serial bus (USB) drive) if the planning computer 214 is located outside the OR.

The device computer 216 in some inventive embodiments is housed in the moveable base 208 and contains hardware, software, data and utilities that are preferably dedicated to the operation of the surgical device 202. This may include surgical device control, robotic manipulator control, the processing of kinematic and inverse kinematic data, the execution of registration algorithms, the execution of calibration routines, the execution of operational data (e.g., cut-files, the trajectory parameters), coordinate transformation processing, providing workflow instructions to a user, and utilizing position and orientation (POSE) data from the tracking system 206. The device computer 216 may further execute one or more of the method steps described above. For example, the device computer 216 may compile the force data to generate the actual force profiles during implant insertion and then compare, in real-time, those actual force profiles to the expected force profile models to determine the quality of the implant placement. The device computer 216 may further provide instructions to the user to improve the quality of the implant placement based on the comparison, wherein the instructions are relayed to the user via the monitor 218.

The tracking system 206 of the surgical system 200 includes two or more optical receivers 230 to detect the position of fiducial markers (e.g., retroreflective spheres, active light emitting diodes (LEDs)) uniquely arranged on rigid bodies. The fiducial markers arranged on a rigid body are collectively referred to as a fiducial marker array 232 and encompass 232a, 232b, 232c, and 232d, where each fiducial marker array of 232 has a unique arrangement of fiducial markers, or a unique transmitting wavelength/frequency if the markers are active LEDs. An example of an optical tracking system is described in U.S. Pat. No. 6,061,644. The tracking system 206 may be built into a surgical light, located on a boom, a stand 234, or built into the walls or ceilings of the OR. The tracking system computer 236 may include tracking hardware, software, data and utilities to determine the POSE of objects (e.g., bones B, surgical device 202) in a local or global coordinate frame. The POSE of the objects is collectively referred to herein as POSE data, where this POSE data may be communicated to the device computer 216 through a wired or wireless connection. Alternatively, the device computer 216 may determine the POSE data using the position of the fiducial markers detected from the optical receivers 230 directly.

The POSE data is determined using the position data detected from the optical receivers 230 and operations/processes such as image processing, image filtering, triangulation algorithms, geometric relationship processing, registration algorithms, calibration algorithms, and coordinate transformation processing. For example, the POSE of a digitizer probe 238 with an attached probe fiducial marker array 232d may be calibrated such that the probe tip is continuously known as described in U.S. Pat. No. 7,043,961. The POSE of the tool tip or tool axis of the tool 124 may be known with respect to a device fiducial marker array 232 using a calibration method as described in U.S. Pat. Pub. US2018/0014888. It should be appreciated that even though the device fiducial marker 232 is depicted on the manipulator arm 210, it may also be positioned on the base 208 or the end-effector 120. Registration algorithms may be executed to determine the POSE and coordinate transforms between a bone B, pre-operative bone data, a fiducial marker array 232a or 232c, and a surgical plan, using the registration methods described above.

The POSE data is used by the computing system 204 during the procedure to update the POSE and/or coordinate transforms of the bone B, the surgical plan, and the surgical robot 202 as the manipulator arm 210 and/or bone B move during the procedure, such that the surgical robot 202 can accurately execute the surgical plan. In another inventive embodiment, the surgical system 200 does not include a tracking system 206, but instead employs a mechanical arm 205, and a bone fixation and monitoring system that fixes the bone directly to the surgical robot 202 and monitors bone movement as described in U.S. Pat. No. 5,086,401.

Other Inventive Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A method to assess the quality of implant placement intra-operatively with a robotic surgical system, comprising:
generating a surgical plan having: a position and orientation (POSE) of a cavity to be created in a bone to receive an implant, and trajectory parameters for robot insertion of the implant;
creating the cavity in the bone;
robotically inserting the implant into the cavity while recording force data;
compiling the force data to generate an actual force profile representing the forces experienced on the implant during insertion; and
comparing the actual force profile to an expected force profile model to determine at least one of: an accuracy of the created cavity, a quality of fit of the implant in the cavity, or an impending bone fracture.

2. The method of claim 1 wherein the cavity is created with the robotic surgical system and the bone is registered to the surgical plan and the surgical system prior to creating the cavity.

3. The method of claim 1 further comprising assembling the implant to an end-effector of the robotic surgical system and determining the POSE of the implant relative to the trajectory parameters in the surgical plan.

4. The method of claim 3 wherein the POSE of the implant is determined by digitizing several unique points on the implant.

5. The method of claim 3 wherein the POSE of the implant is determined by registering the implant to a model of the implant stored in the robotic surgical system.

6. The method of claim 1 wherein the POSE of the cavity to be created in the bone is determined based on the POSE of an implant model in a bone model.

7. The method of claim 6 wherein the trajectory parameters comprise a set of vectors, lines, points, or a combination thereof, the trajectory parameters defined relative to the bone model.

8. The method of claim 1 further comprising comparing the force data during insertion to a preselected threshold force and pausing robotic insertion when the preselected threshold force is exceeded, and optionally warning the user of an impending bone fracture.

9. The method of claim 1 further comprising improving the fit of the implant based on the comparison by: adjusting the dimensions of the cavity; or adding, removing, or adjusting one or more implant components or adjuncts.

10. The method of claim 1 wherein the expected force profile model is generated by:
generating a surgical plan on a mock bone, said plan having: a POSE of a cavity to be created in the mock bone, and trajectory parameters for robotic insertion of the implant;
creating the cavity in the mock bone;
robotically inserting the implant into the cavity while acquiring force data;
compiling the force data into a force profile;
conducting one or more tests on the implant in the cavity to determine an acceptability of the implant fit; and
building the expected force profile model using force profiles from several mock bone cases having an acceptable implant fit.

11. The method of claim 10 wherein the one or more tests include at least one of:
longevity testing, micro-motion studies, imaging scans, load testing, stress analysis, fatigue testing, or finite element analysis with the data from the imaging scans.

12. The method of claim 10 wherein the one or more tests include Pass/Fail criteria to determine the acceptability of the implant fit.

13. The method of claim 10 wherein several expected force profile models are generated each for a specific implant model or size, or for a specific cavity to be created.

14. The method of claim 10 further comprising inserting said implant into the cavity.

15. A robotic surgical system to assess the quality of implant placement intra-operatively, the system comprising:
a surgical robot having a manipulator arm;
an end-effector attached to a distal end of the manipulator arm;
an implant attached to the end-effector;
a surgical plan having trajectory parameters to guide the manipulator arm to robotically insert the implant into a cavity;
a force sensor to sense forces generated on the implant during robotic insertion; and
a computer having a processor and memory, said memory having an expected force profile model stored therein in communication with a comparison module that when executed by the processor causes the processor to build an actual force profile from force data collected from the force sensor during robotic insertion and compare the actual force profile to the expected force profile model to determine at least one of an accuracy of the created cavity; a quality of fit of the implant in the bone; or an impending bone fracture.

16. The system of claim 15 further comprising a display monitor, wherein the comparison module when executed by the processor further causes the processor to determine a set of instructions to improve the quality of the implant placement and display said instructions on the monitor.

17. The system of claim 15 wherein the end-effector is an implant holder configured to mechanical engage with the implant.

18. The system of claim 17 wherein the implant holder is at least one of: a pair of jaws, clamps, or fingers for grasping the implant; a magnet to magnetically couple with the implant; a vacuum; an adhesive to stick to the implant; a screw or pin that assembles with a corresponding feature on the implant; or other fastening element or coupler.

19. The system of claim 17 wherein the end-effector further includes a tool holder, wherein the implant holder is assembled to the tool holder.

20. The system of claim 15 wherein the surgical plan further comprises a position and orientation (POSE) for the cavity to be created in the bone, wherein the end-effector includes an end-mill to robotically create the cavity in the bone.
